(19) United States
(12) Patent Application Publication
(10) Pub. No.: US 2010/0184946 A1
(43) Pub. Date: Jul. 22, 2010

(54) Preparation of Fine Particles

(76) Inventor: Huibert Albertus Van Boxtel, TK Tilburg (NL)

Correspondence Address: MORGAN LEWIS & BOCKIUS LLP, 1111 PENNSYLVANIA AVENUE NW, WASHINGTON, DC, 20004, US

(21) Appl. No.: 12/669,250

(22) PCT Filed: Jul. 17, 2008
(86) PCT No.: PCT/GB08/02450

§ 371 Date: Feb. 23, 2010

(30) Foreign Application Priority Data
Jul. 20, 2007 (GB) 0714223.5

(51) Int. Cl. C07K 7/64; C07D 305/14; C07C 69/67; B01J 19/18

(52) U.S. Cl. 530/317; 549/510; 560/52; 422/259

(57) ABSTRACT

A process and device for the precipitation of an organic compound comprising the steps:
(a) providing a first stream comprising an organic compound and a solvent for the organic compound;
(b) providing a second stream comprising an anti-solvent for the organic compound;
(c) providing a third stream comprising a second stabilising agent;
(d) intermixing the first and second streams to form a precipitate of the organic compound in particulate form; and
(e) following step (d), intermixing the third stream with the intermixed first and second streams containing the precipitated organic compound in particulate form
   wherein the first and/or the second stream comprises a first stabilising agent.

This invention relates to a process for the precipitation of organic compounds in particulate form and to a device suitable for use in such a process.

In the pharmaceuticals field, there are many factors which can affect the bioavailability of drugs and therefore their effectiveness at treating diseases and medical disorders. These factors include the particle size, the particle size distribution and the dissolution rate of the active ingredient. Poor bioavailability is a significant problem encountered in the development of pharmaceutical compositions, particularly those containing an active ingredient that is poorly soluble in water. Poorly water-soluble drugs, e.g. those having a solubility less than about 10 mg/ml, tend to be eliminated from the gastrointestinal tract before being absorbed into the circulation. Moreover, poorly water soluble drugs can give rise to difficulties when required for intravenous administration in terms of blocking needles and even blocking tiny blood vessels in patients.

It is known that the rate of dissolution of particulate drugs can increase with increasing surface area, e.g. by decreasing particle size, and by decreasing the degree of crystallinity. Consequently, methods of making finely divided drugs have been studied and efforts have been made to control the size and size range of drug particles in pharmaceutical compositions. For example, dry milling techniques have been used to reduce particle size and hence influence drug absorption. However, in conventional dry milling, the limit of fineness is often in the region of 100 microns (100,000 nm) when material begins to cake on the walls of the milling chamber. Wet grinding is beneficial in further reducing particle size, but flocculation restricts the lower particle size limit in many cases to approximately 10 microns (10,000 nm).

U.S. Pat. No. 4,826,689 describes a method for making particles of water-insoluble drugs comprising the slow infusion of water into a solution of the drug in an organic solvent. The water, which acts as an anti-solvent, may contain a surfactant, e.g. Pluronic F-68 or a gelatine. This batch-wise process appears to be quite slow and laborious. Many of the resultant particles were not particularly small. The resultant particles did not appear to be particularly stable either because they still had to be promptly separated from the organic solvents to prevent redissolving and reprecipitation of particles at undesirable sizes. This created time pressures that would not exist if the particles were more stable.

US patent application publication no. 2005/0202095 A1 describes an alternative process for making fine particles by mixing an anti-solvent and a solvent containing the desired compound in an off-the-shelf rotor stator device such as a Silverson Model L4RT-A Rotor-Stator. However the resultant particles were very large, e.g. in the Examples the precipitated glycine particles ranged in size from 4.4 microns to 300 microns.

US patent application no. 2007/071825 describes a device of the rotor/stator type for continuously producing particles by feeding an organic phase and an aqueous phase to a homogenization compartment. Particles are produced in a very wide size range and no data are disclosed about the storage stability and the redispersibility of the particles.

There exists a need for a process for preparing organic compounds, particularly pharmaceutical actives, with a small particle size without the need for potentially wasteful and damaging milling and without the need for accurately positioned jets which might clog. Ideally the process is operable on the industrial scale, is rapid, not unduly complicated and leads to small particles which can be dried into a stable dry product to enhance their shelf life. Also it is desirable for the dried product to be readily redispersible in water or an aqueous solvent at ambient temperature prior to administration, to form a suspension with a similar particle size distribution to that prior to drying. Ideally the resultant particles are stable to the extent that the manufacture is not rushed to remove them from the liquid medium in which they are formed in order to avoid undesirable redissolving or reprecipitation of particles at undesirable sizes.

According to the present invention there is provided a process for the precipitation of an organic compound in particulate form comprising the steps:
(a) providing a first stream comprising an organic compound and a solvent for the organic compound;
(b) providing a second stream comprising an anti-solvent for the organic compound;
(c) providing a third stream comprising a second stabilising agent;
(d) intermixing the first and second streams to form a precipitate of the organic compound in particulate form; and
(e) following step (d), intermixing the third stream with the intermixed first and second streams;
   wherein the first and/or the second stream comprises a first stabilising agent.

In this document (including its claims), the verb “comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The particles obtained by the process of this invention can be in any form, including amorphous and crystalline forms, polymorphs, hydrates and solvates, as well as salts including addition salts, with amorphous particles being greatly preferred.

The organic compound is preferably an organic pharmaceutically active compound, a dye or an agrochemical. The organic compound may also be an organometallic compound, e.g. haemoglobin or an organic compound in salt form.

Examples of organic compounds include “biological” organic compounds such as hormones, proteins, peptides, carbohydrates, amino acids, lipids, vitamins, enzymes and the like.

The organic compounds which can be precipitated according to the method of this invention are preferably pharmaceutically active organic compounds. Classes of such compounds include anabolic steroids, analeptics, analgesics, anaesthetics, antacids, anti-arrythmics, anti-asthmatics, antibiotics, anti-carcinogenics, anti-cancer drugs, anticoagulants, anticofonergics, anticonvulsants, antidepressants, anti-diabetics, anti-diarrhoeals, anti-emetics, anti-epileptics, antifungals, antihelmintics, anti-hemorrhoidals, antihistamines, antihormones, anti-hypertensives, anti-hypotensives, anti-inflammatories, antimuscarinics, antimycotics, antineoplastics, anti-obesity drugs, antiplaque agents, antiprotozoals, antipsychotics, antiseptics, anti-spasmotics, anti-thrombics, antitussives, antivirals, anxiolytics, astringents, beta-adrenergic receptor blocking drugs, bile acids, breath fresheners, bronchospasmolytic drugs, bronchodilators, calcium channel blockers, cardiac glycosides, contraceptives, corticosteroids, decongestants, diagnostics, digestives, diuretics, dopaminergics, electrolytes, emetics, expectorants, haemostatic drugs, hormones, hormone replacement therapy drugs, hypnotics, hypoglycaemic drugs, immunosuppressants, impotence drugs, laxatives, lipid regulators, mucolytics, muscle relaxants, non-steroidal anti-inflammatories, nutraceuticals, pain relievers, parasympathicolytics, parasympathicomimetics, prostaglandins, psychostimulants, psychotropics, sedatives, sex steroids, spasmolytics, steroids, stimulants, sulfonamides, sympathicolytics, sympathicomimetics, sympathomimetics, thyreomimetics, thyreostatic drugs, vasodilators, vitamins, xanthines, and mixtures thereof. A particularly preferred organic compound from the class of anti-cancer drugs is paclitaxel (also known as Taxol).

While the process is particularly useful for preparing pharmaceutical active compounds in a particulate form, it may also be used to provide particles of other organic compounds, for example agrochemicals, colorants, cosmetics and the like.

Preferably the precipitated organic compound arising from the process has an average particle size of less than 1 micron, more preferably less than 700 nm, especially less than 500 nm, more especially less than 150 nm. Preferably the precipitated organic compound has a unimodal particle size distribution.

For organic compounds which are pharmaceutically active, the optimal reported size for long circulating nanoparticles that trap in tumour tissue varies. It can be, for example, 100 to 200 nm or even less than 100 nm. The optimum size for endocytosis (cellular absorption) is believed to be 100 to 200 nm.

Preferably the precipitated organic compound in particulate form has a D50 of less than 500 nm, more preferably less than 400 nm, especially less than 300 nm, more especially less than 200 nm. The D50 may be measured by techniques known in the art, for example by Laser diffraction using the method according to ISO 13320-1, e.g. using a Malvern Mastersizer 2000 particle size analyser.

Preferably the first stream comprises the first stabilising agent because it is this stream which contains the organic compound requiring stabilisation and the stabilisation of the organic compound is generally improved compared to the case when the first stream does not contain any stabilising agent.

The amounts of each component in the first stream may be varied between wide limits and depend to some extent on the properties of the components and the properties of the other streams they come into contact with. Typically however the first stream will comprise 0.1 to 50 wt %, more preferably 0.2 to 10 wt % of organic compound, relative to the weight of solvent. The amount of stabilising agent included in the first stream is typically 0.1 to 50 wt %, more preferably 0.2 to 20 wt % relative to the weight of solvent.

While not wishing to be limited to any particular theory, the stabilisation agents are believed to be useful in the present process in a number of ways. For example, a stabilisation agent may inhibit particle agglomeration by surface adsorption and steric repulsion of particles. A stabilisation agent may lower the particle growth rate by polymer adsorption to the particle surface. The stabilisation agents may even increase viscosity due to the polymer presence, thereby limiting the agglomeration rates.

The stabilising agents which are included in the first and third streams, or the second and third, or the first, second and third streams may be identical but preferably they are not identical. Preferably the stabilising agents are polymeric stabilising agents. Preferably at least one of the stabilising agents is an amphiphilic polymer, more preferably amphiphilic block copolymer, or a mixture comprising such a polymer or copolymer. For organic compounds intended for use in the pharmaceutical preparations the stabilising agents are preferably biocompatible amphiphilic block copolymers.

The amphiphilic polymers, and in fact the stabilising agents in general, preferably have an affinity for both the organic compound and the anti-solvent e.g. water. When the organic compound has a low solubility in water, the amphiphilic polymer will generally possess a hydrophilic part which has an affinity for water and a less hydrophilic part, e.g. a relatively hydrophobic part, which has an affinity for the organic compound. The relatively hydrophilic part of the amphiphilic polymers are often non-ionic (e.g. polyethylene oxide units) and/or ionic (e.g. they have anionic or cationically charged groups) while the less hydrophilic or hydrophobic parts are often electrically neutral and relatively non-polar (e.g. polylactide groups).

Preferred amphiphilic copolymers which are not block copolymers include gelatines, especially gelatines having a molecular weight of at least 2 kDa. Many gelatine solutions gel at temperatures at or below room temperature. The suspension stability of particles prepared by the process of the present invention is in some cases greatly enhanced at temperatures below room temperature.

Therefore in one embodiment the use of non-gelling gelatines is preferred over the use of gelling gelatines. Examples of such gelatines include fish gelatines, non-gelling recombinant gelatines, e.g. recombinant gelatines without hydroxyproline, and hydrolysed gelatines with very low molecular weight. In one embodiment the gelatine shows no gelling properties when stored as a 2 wt % solution in water at 10° C. for 4 hours. Particularly preferred gelatines are recombinant gelatines, especially where the particles are intended for use in vivo (e.g. in pharmaceutical applications) due to the absence of BSE or viral risks.

In a preferred embodiment the first stabilising agent comprises an amphiphilic block copolymer and the second stabilising agent comprises an amphiphilic copolymer, for example the second stabilising agent comprises a gelatine. In some embodiments the second stabilising agent may be an amphiphilic block copolymer too.

The preferable block-type and block-lengths in amphiphilic block copolymers can vary depending on the chemical composition of the first and second streams and on the preferred average particle size after precipitation. Preferably the amphiphilic polymer comprises hydrophilic and relatively hydrophobic segments. Preferably the amphiphilic polymers are triblock and diblock copolymers, especially diblock copolymers. Typically such copolymers comprise at least one hydrophilic block and at least one relatively hydrophobic block.

The polymeric stabilizers are preferably biocompatible, especially where the organic compound is a pharmaceutical compound.

Considering the aforementioned, preferred hydrophilic blocks are poly(ethylene glycol) (“PEG”) and/or poly(ethylene glycol) monoether (“PEG ether”) blocks. One of the reasons for preferring PEG and PEG ether hydrophilic blocks is because stabilisation agents containing these blocks tend to be excreted from the body less quickly than stabilisation agents lacking these blocks. So where the organic compound is, for example, an anti-cancer drug the presence of PEG and PEG ether hydrophilic blocks in the stabilisation agent can increase the time the drug is in the body making it more effective.

Furthermore, first stabilizers comprising amphiphilic block copolymers having a number average molecular weight (Mₙ) below 10,000 generally have better stabilising properties than those of higher Mₙ.

The preferred ethers have from 1 to 4 carbon atoms, with methyl ether being most preferred. Preferred hydrophobic blocks are poly(lactic-co-glycolic)acid (“PLGA”), poly(styrene) (“PS”), poly(butyl acrylate), poly(ε-caprolactone) and especially polylactide (“PLA”) blocks. Polylactides are polyesters formed from the polymerisation of lactic acid or lactide. Polylactides exist as poly-L-lactide, poly-D-lactide and poly D,L-lactide.

Preferred biocompatible amphiphilic block copolymers include copolymers comprising one or more PEG and/or PEG ether blocks and one or more polylactide (“PLA”) blocks. The PEG blocks are relatively hydrophilic compared to the PLA blocks.

In one embodiment it is preferred that the PEG and PEG ether block(s) have an Mₙ of 250 to 5000, more preferably 400 to 4000, especially 500 to 2000, more especially 600 to 1500. Very good results were obtained with a PEG having an Mn of 750.

In another embodiment it is preferred that the PEG and PEG ether block(s) have a number weighted average molecular weight (Mₙ) of 250 to 5000, more preferably 500 to 4000, especially 1000 to 3000. Very good results were obtained with a PEG and PEG ether blocks having an Mn of about 2000.

Thus in a preferred process according to the invention the amphiphilic copolymer is an amphiphilic block copolymer comprising a PEG Mₙ 250-5000 block and/or a PEG Mₙ, 250-5000 (C₁₋₄-alkyl)ether block, with the preferred Mn of such block(s) being 400 to 4000, especially 500 to 2000, more especially 600 to 1500, and particularly 750. In another preferred process according to the invention the amphiphilic copolymer is an amphiphilic block copolymer comprising a PEG block of Mₙ 250-5000 and/or a (C₁₋₄-alkyl)ether of a PEG block of Mᵣ, 250-5000, with the preferred Mn of such block(s) being 500 to 4000, especially 1000 to 3000, and particularly about 2000.

In a preferred embodiment the PLA block(s) have an Mₙ, of 250 to 5000, more preferably 400 to 4000, especially 500 to 2000 and more especially from 600 to 1500. Very good results were obtained with a PLA block having an Mn of 1000.

In another embodiment it is preferred that the PLA block(s) have an Mₙ of 250 to 5000, more preferably 500 to 4000, especially 1000 to 3000. Very good results were obtained with a PLA block having an Mn of about 2000.

A particularly preferred amphiphilic block copolymer is a diblock copolymer of a PEG ether and a PLA, especially having the Mₙ mentioned above, with the preferences for Mₙ, in each block being as mentioned above. Bearing in mind the above, one preferred category of amphiphilic diblock copolymers are poly(ethylene glycol) Mₙ 350-5000 (C₁₋₄-alkyl)ether-block-polylactide Mₙ 1000-5000. Examples of valuable subsets of such amphiphilic diblock copolymers include:

poly(ethylene glycol) Mₙ 350-1500 (C₁₋₄-alkyl)ether-block-polylactide Mᵣ, 500-2000;
poly(ethylene glycol) Mₙ 500-1100 (C₁₋₄-alkyl)ether-block-polylactide Mₙ 600-1600;
poly(ethylene glycol) Mₙ 600-900 (C₁₋₄-alkyl)ether-block-polylactide Mₙ, 800-1200;
poly(ethylene glycol) Mₙ 700-900 (C₁₋₄-alkyl)ether-block-polylactide Mₙ 800-1200;
poly(ethylene glycol) Mₙ, 700-900 methyl ether-block-polylactide Mₙ, 800-1200;
poly(ethylene glycol) Mₙ, 750 (C₁₋₄-alkyl)ether-block-polylactide Mₙ, 1000; and
poly(ethylene glycol) Mₙ 750 methyl ether-block-polylactide Mₙ 1000. Examples of amphiphilic block copolymers include:
poly(ethylene glycol) Mₙ 750 mono methyl ether-block-polylactide methyl ether Mₙ 1000;
poly(ethylene glycol) Mₙ 2000 mono methyl ether-block-polylactide methyl ether Mₙ 2000;
poly(ethylene glycol) Mₙ 3000 mono methyl ether-block-polylactide methyl ether Mₙ 2000;
poly(ethylene glycol) Mₙ 350 methyl ether-block-polylactide Mₙ 1000;
poly(ethylene glycol) Mₙ 5000 methyl ether-block-poly(lactone) Mₙ ˜5,000;
poly(ethylene glycol) Mₙ 5000 methyl ether-block-poly(ε-caprolactone) Mₙ 5,000;
poly(ethylene glycol) Mₙ 5000 methyl ether-block-poly(ε-caprolactone) Mₙ 13,000; and
poly(ethylene glycol) Mₙ 5,000 methyl ether-block-poly(ε-caprolactone) Mₙ 32,000;
all of which are commercially available from Sigma-Aldrich Co. As will be readily understood by those skilled in the art, “methyl ether” refers to a methyl group on one end of the PEG chain (not both ends because this would prevent the PLA from attaching to the PEG). Also the Mn values for the PEG, such in “PEG mono methyl ether Mn 750” refer to the Mn of the PEG per se, not including the extra CH₂ group of the methyl group.

Amphiphilic polymers are available from commercial sources or they may be synthesised ad hoc for use in the process. The preparation of the preferred amphiphilic diblock copolymers with poly(alkylene glycol) (PAG) blocks (e.g. poly(ethylene glycol) (PEG) blocks) can be performed in a number of ways. Methods include: (i) reacting a hydrophobic polymer with methoxy poly(alkylene glycol), e.g. methoxy PEG or PEG protected with another oxygen protecting group (such that one terminal hydroxyl group is protected and the other is free to react with the hydrophobic polymer); or (ii) polymerizing the hydrophobic polymer onto methoxy or otherwise monoprotected FAG, such as monoprotected PEG. Several publications teach how to carry out the latter type of reaction. Multiblock polymers have been prepared by bulk copolymerization of D,L-lactide and PEG at 170°-200° C. (X. M. Deng, et al., J. of Polymer Science: Part C: Polymer Letters, 28, 411-416 (1990). Three and four arm star PEG-PLA copolymers have been made by polymerization of lactide onto star PEG at 160° C. in the presence of stannous octoate as initiator. K. J. Zhu, et al., J. Polym. Sci., Polym. Lett. Ed., 24, 331 (1986), “Preparation, characterization and properties of polylactide (PLA)-poly(ethylene glycol) (PEG) copolymers: a potential drug carrier”. Triblock copolymers of PLA-PEG-PLA have been synthesized by ring opening polymerization at 180-190° C. from D,L-lactide in the presence of PEG containing two end hydroxyl groups using stannous octoate as catalyst, without the use of solvent. The polydispersity (ratio Mw to Mn) was in the range of 2 to 3.

In an alternative embodiment, the hydrophobic polymer or monomers can be reacted with a poly(alkylene glycol) that is terminated with an amino function (available from Shearwater Polymers, Inc.) to form an amide linkage, which is in general stronger than an ester linkage.

Triblock or other types of block amphiphilic copolymers terminated with poly(alkylene glycol), and in particular, poly(ethylene glycol), can be prepared using the reactions described above, using a branched or other suitable poly(alkylene glycol) and protecting the terminal groups that are not to be reacted. Shearwater Polymers, Inc., provides a wide variety of poly(alkylene glycol) derivatives. Examples are the triblock PEG-PLGA-PEG.

In one embodiment, a multiblock amphiphilic copolymer is used and this may be prepared by reacting the terminal group of the hydrophobic polymeric block such as PLA or PLGA with a suitable polycarboxylic acid monomer, for example 1,3,5-benzenetricarboxylic acid, butane-1,1,4-tricarboxylic acid, tricarballylic acid (propane-1,2,3-tricarboxylic acid), and butane-1,2,3,4-tetracarboxylic acid, wherein the carboxylic acid groups not intended for reaction are protected by means known to those skilled in the art. The protecting groups are then removed, and the remaining carboxylic acid groups reacted with poly(alkylene glycol). In another alternative embodiment, a di, tri, or polyamine is similarly used as a branching agent.

Preferably the first stream and/or the second stream contains a stabilising agent for the organic compound. Depending on the solubility of the first stabilising agent it can be dissolved in the first stream or the second stream or both.

The solvent may be any liquid in which the organic compound is soluble or dispersible. The solvent may be, for example, polar or non-polar, protic or aprotic, ionic or non-ionic. Preferably however the solvent is or comprises an organic solvent. Preferably the solvent and the antisolvent are miscible. Preferably the solvent comprises one or more water-miscible organic solvents. Preferably the solvent is such that the organic compound has a high solubility therein, preferably a solubility when measured at 20° C. of at least 10 g/l.

The first stream comprising the organic compound may comprise a single solvent or a mixture of solvents. The anti-solvent for the organic compound is preferably a liquid in which the organic compound has a solubility of less than 1 wt %, more preferably less than 0.1 wt %, at a temperature of 20° C. and a pressure of 1 bar. Preferably the anti-solvent comprises water. If solubility allows, the second stream may comprise a first stabilising agent. The anti-solvent used in the second stream may be chosen to suit the components of the first stream, i.e. the organic compound, the solvent and the first stabilising agent. The particular conditions used for the process may also influence the decision on which anti-solvent to use. The second stream comprising the anti-solvent may, for example, be a liquid having a lower temperature (in case of low temperature precipitation), different ionic strength or different pH than the first stream. The second stream may comprise one anti-solvent or more than one anti-solvent. Examples of anti-solvents include water, alcohols and liquid alkanes. Whether or not the specific liquid is an anti-solvent depends on the organic compound that needs to be precipitated. In addition the second stream may also contain a solvent for the organic compound, although this would generally be present in only small amounts so as not to adversely affect the ability of the second stream to cause the organic compound to precipitate when the first and second streams are mixed. In one embodiment the first and/or second stream comprises a wetting agent.

In step (d) the first stream may be fed into the flow of the second stream or the second stream may be fed into the flow of the first stream. In other words the terms “first stream” and “second stream” are not intended to imply any particular order but merely identify the particular two streams being referred to.

In a preferred process according the present invention the second stream further comprises a peptising agent, e.g. citric acid. The citric acid may be in the free acid or salt form.

The amounts of peptising agent in the second stream is typically 0 to 5 wt %, more preferably 0.1 to 2 wt %, relative to the weight of anti-solvent.

Preferably the second stream has a faster flow rate than the first stream. In one embodiment the second stream has a flow rate of 1.5 to 10, more preferably 2 to 7 times the flow rate of the first stream. Preferably however the second stream has a flow rate of 1.5 to 50, more preferably 2 to 20, especially preferably 3 to 8 and more especially about 5 times the flow rate of the first stream.

The third stream comprises a second stabilising agent and preferably an anti-solvent for the organic compound. Examples of anti-solvents and stabilising agents are mentioned above. The anti-solvent in the third stream may be different from the anti-solvent used in the second stream, although preferably it is the same. The stabilising agents in the third stream may be the some as the stabilising agents used in the first and/or second stream, although preferably they are different.

The amount of second stabilising agent in the third stream is typically 0.1 to 50 wt %, more preferably 1 to 25 wt %, relative to the weight of the third stream.

The third stream can have a lower, a similar or a faster flow rate than the intermixed first and second streams. Preferably however the third stream has a flow rate similar or somewhat higher than that of the intermixed first and second streams. For example, good results are obtained with a flow rate ratio of third stream of 0.1 to 10, more preferably 0.5 to 10, especially 0.5 to 5 times the flow rate of the intermixed first and second streams.

One or more of the first, second and third streams may contain a wetting agent if desired. In one embodiment the wetting agent is biocompatible. This is especially useful when the organic compound is a pharmaceutically active compound. Preferred wetting agents include sodium dodecylsulphate. Tween 80, Cremophor A25, Cremophor EL, Pluronic F68, Pluronic L62, Pluronic F88, Span 20, Tween 20, Cetomacrogol 1000, Sodium Lauryl Sulphate, Pluronic F127, Brij 78, Klucel, Plasdone K90, Methocel E5, PEG, Triton X100, Witconol-14F and Enthos D70-30C.

Biocompatible wetting agents include polyethoxylated castor oils, for example Cremophor EL.

The intermixing may be achieved by a number of means, for example turbulent flow, sonication and/or mixing using a mechanical stirring means. Preferably the intermixing comprises the use of a mechanical stirring means, for example as described in more detail below.

In a preferred embodiment the process steps (a) to (e) are performed in a continuous manner.

Following step (e) the precipitated, stabilised particulate organic compound may be collected in a continuous or batchwise manner.

If desired the process may also include the step of drying the precipitated organic compound, for example using a spray drier and/or freeze drying. Drying is often useful to provide good storage stability and typically entails removal of organic solvents and anti-solvents.

During freeze drying, the precipitated organic compound together with any solvent and anti-solvent are cooled and subjected to a reduced pressure. As a result the solvent and anti-solvent are removed from the precipitated organic compound by evaporation under very mild conditions which do not adversely effect the precipitated organic compound.

The freezing may be performed using dry ice or, more preferably, liquid nitrogen, In a preferred embodiment freeze drying comprises adding: the precipitated organic compound together with any solvent and water which may be present to liquid nitrogen, preferably in a dropwise manner, followed by removal of the solvent and anti-solvent by evaporation using a pressure below atmospheric pressure. This technique generally results in little if any damage to the particles of precipitated organic compound and provides a physical form which may be handled easily and safely.

Still further the process optionally further comprises the step of re-dispersing the dried precipitate in a liquid medium.

The process of the present invention may be performed on any scale and steps (a) to (e) may be performed in a continuous manner. In this way large quantities of the desired particulate organic compound may be prepared, including on an industrial scale. There is no need to include jets in the process which have to be carefully aligned. The conditions may be tailored to give small particles which can be isolated and redispersed without difficulty.

In one embodiment, step (d) is performed in a first mixing chamber and step (e) is performed in a second mixing chamber. In another embodiment, step (d) and step (e) are performed in the same mixing chamber.

In another preferred embodiment, step (d) is performed in a closed type mixing chamber and step (e) is performed in a closed type mixing chamber which is the same chamber or a different chamber from that used in step (d).

In a particularly preferred embodiment of the process:
i. the first stream comprises an organic compound, a water-miscible organic solvent for the organic compound and a first stabilising agent comprising an amphiphilic copolymer;
ii. the second stream comprises water and optionally citric acid;
iii. the third stream comprises water and a second stabilising agent comprising an amphiphilic copolymer; and
iv. the first stabilising agent is not identical to the second stabilising agent.

After the process has begun, a steady state may be reached in which the first and second streams are continuously fed into a precipitation chamber and after step (d) the precipitate of organic compound in particulate form is fed continuously into a stabilisation chamber where it is intermixed with a the third stream comprising the second stabilising agent.

Preferably, the residence time in each of the precipitation and stabilisation chamber is more than 0.0001 second and less than 5 seconds, preferably more than 0.001 second and less than 3 seconds. When the residence time in the precipitation chamber is too long, extremely fine grains formed therein may grow to larger sizes before they have been stabilised in the stabilisation chamber and the average particle size distribution may become undesirably wide. When the residence time is too short, too few nuclei may be formed. The optimum residence time will vary from one organic compound to another and may be optimised by simple trial and error.

As mentioned above, the intermixing maybe performed in various manners. The preferred method for intermixing comprises mixing using a mechanical stirring means, which can be driven in any way, for example by a drive shaft or by a rotating magnet. Preferably the mechanical stirring means is rotatable within a mixing chamber, for example it may comprise a rotatable blade. When a “mixing chamber” is referred to here this is used generically to encompass the precipitation chamber and a stabilisation chamber where steps (d) and (e) respectively may be performed, unless the context implies otherwise. The blade may be in any form and have any aspect ratio, for example it may be in the form of a paddle where the ratio of its height to width are similar, or it may be in the form of disc, e.g. its height is very much smaller than its width. By width we mean twice the diametric distance from the central axis of rotation of the paddle to its outermost edge. It is preferred that the volume of the mechanical stirring means is at least 10% and not more than 99%, more preferably at least 15% and not more than 95% of the volume of the relevant mixing chamber. The mechanical stirring means preferably comprises a shaft and stirrer blade which may be rotated by the shaft. A preferred size of stirrer blade is at least 50%, more preferably at least 70%, especially 80% to 99% and a more especially 80% to 95% of the smallest diameter of the relevant mixing chamber.

To assist with the intermixing in step (d) and (e) it is preferred that the precipitate of the organic compound and the liquid phase is discharged from the mixing chamber(s) through an outlet which is towards the opposite end of the mixing chamber from the inlets and not directly in line with the inlets. For example, the inlets may be positioned at the bottom part of the mixing chamber and the outlet(s) may be positioned at the top part of the mixing chamber. In one embodiment the inlets are below the middle line of the mixing chamber (e.g. below 30% height or 20% height). The outlet(s) may be above 70% height. In another embodiment, the outlet(s) is or are approximately at a right angle (e.g. 80 to 100° angle, especially 90° angle) relative to the flow of streams through the inlets. In this way the streams entering through the inlets do not immediately exit through the outlet without proper intermixing.

In one embodiment the mixing chambers have more than one outlet.

The precipitate arising from step (e) is preferably discharged into a collecting vessel. The collecting vessel may comprise a second liquid phase comprising one or more of stabilisation agents, wetting agents, non-solvents, solvents or mixtures thereof.

In another embodiment, ripening of the precipitate of the organic compound is performed in a collecting vessel until the preferred average particle size and/or average particle size distribution is achieved. This modification or ripening can be achieved by stirring the product of step (e) in a collecting vessel. During modification or ripening, the average particle size may increase, but the average particle size distribution usually becomes narrower which is sometimes advantageous. Modification or ripening can be controlled by various parameters, e.g. temperature, pH or ionic strength. Consequently, according to this preferred embodiment, the process according to the present invention comprises a further step (f), wherein the product of step (e) is fed into a collecting vessel and subjected to a ripening step.

During an induction period of the process according to the present invention, the second stream comprising the anti-solvent may be introduced with a continuous flow into a precipitation chamber and may travel from there to a stabilisation chamber via fluid communication means (e.g. a pipe) and thereafter to a collecting vessel. Subsequently, the first stream comprising the organic compound may be introduced with a continuous flow into the precipitation chamber where it is intermixed with the second stream which results in a supersaturation of the organic compound thereby initiating the formation of a precipitate and a liquid phase. The term “supersaturation” refers to a concentration of an organic compound that is in excess of saturation under the given conditions, i.e. solvent or solvent mixture, temperature, pH, ionic strength etc. In the liquid phase, the supersaturation may be reduced to such a level that essentially no precipitation will occur outside the precipitation chamber. Then the precipitate may be transferred to the stabilisation chamber where it is intermixed with the third stream. In this embodiment the initial output of the stabilisation chamber may be discarded because it may not contain any precipitate until the steady state has been reached and all streams are flowing. Since in this embodiment the streams are fed continuously, a continuous outflow of the precipitate and the liquid phase is eventually achieved. After the induction period, a steady state is reached in the mixing chambers meaning that basically the composition of the mixture within each mixing chamber is stable and essentially does not change over time. Additionally, the composition of the outflow of the mixing chamber(s) is stable and essentially does not change over time either.

The velocities of the inflow of the various streams do not need to be identical. If multiple inlets are used, the velocity of one stream may differ from the velocity of another stream. However, in general the feed velocity of the streams may be, for example, 0.01 m/s, 0.1 m/s or 1 m/s. Even velocities of 10 m/s or more than 50 m/s can be used. The advantage of this inventive method is, however, that with relatively low stream velocities small particle precipitation can be achieved. Feed velocities in case of multiple inlets need not to be equal. In contrast, in impinging jet mixers it is important and in fact essential that these feed velocities match each other. Strictly speaking it is not the feed velocities but the feed momenta (mass×velocity) for both streams that need to be matched in impinging jet mixers This detail however does not affect the basic argument that the liberty of having unmatched feed velocities or feed momenta in the current invention is a clear advantage. The ratio of feed velocities of first stream to second stream can be 1:99 to 99:1. The ratio of feed velocities of third stream to the combined first and second stream can also be 1:99 to 99:1. During the induction period, the outflow from the stabilisation chamber is collected until the composition of the outflow is essentially constant. As soon as a steady state is reached, the precipitate and the liquid phase may be collected, for example in a collecting vessel.

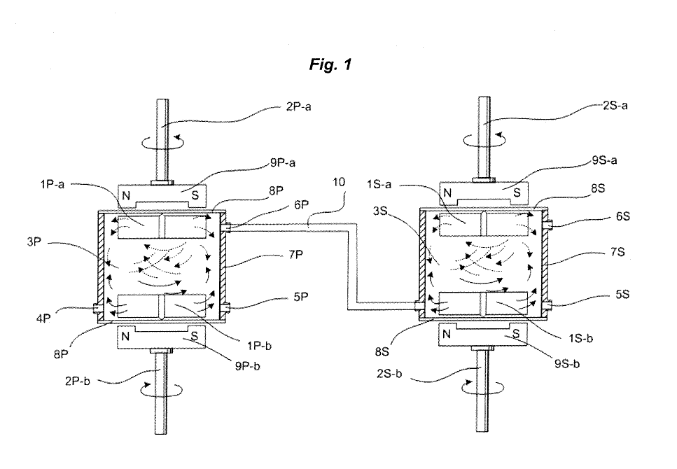

FIG. 1 shows an example of a device according to the present invention which may be used to perform the process.

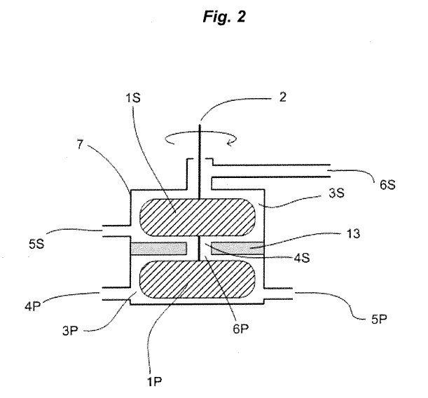

FIG. 2 shows a cross-sectional view of a preferred embodiment of the device.

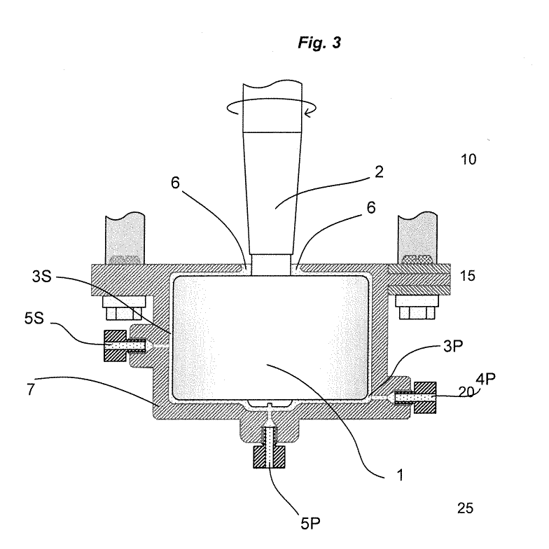

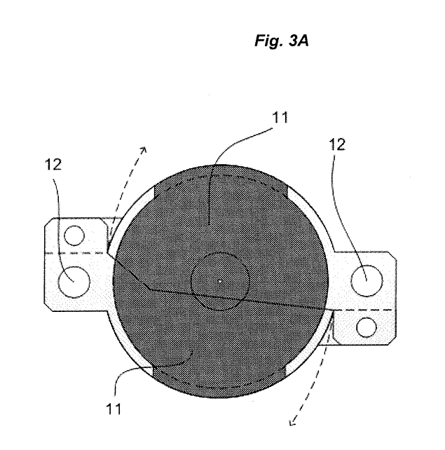

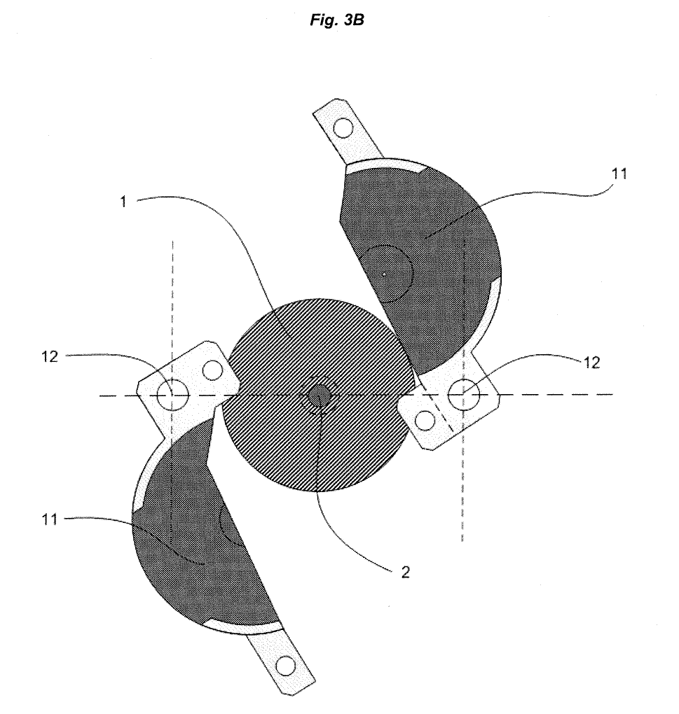

FIG. 3 shows a cross-sectional view of another preferred embodiment of the device.

FIGS. 3A and 3B show top views of a more preferred embodiment of the device shown in FIG. 3.

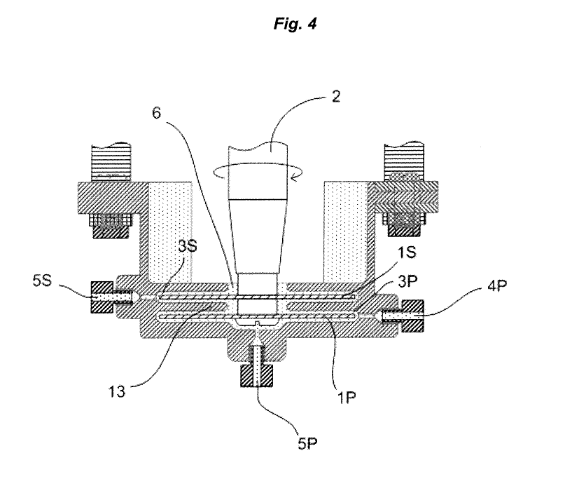

FIG. 4 shows a cross-sectional view of more detailed illustration of the embodiment shown in FIG. 2.

### KEY TO THE SYMBOLS USED IN THE DRAWINGS

1, 1P, 1S, 1P-a, 1P-b, 1S-a, 1S-b: Mechanical stirring means
2, 2P-a, 2P-b, 2S-a, 2S-b: Axis or shaft
3P, 3S: Mixing chamber
4, 5: Inlet
4P: First inlet to the precipitation chamber for the first stream
5P: Second inlet to the precipitation chamber for the second stream
4S: First inlet to the stabilisation chamber to receive the output of the precipitation chamber
5S: Second inlet to the stabilisation chamber for the third stream
6: Outlet
6P: Outlet of the precipitation chamber
6S: Outlet of the stabilisation chamber
7, 7P, 7S: Mixing chamber wall
8P, 8S: Seal plate
9P-a, 9P-b, 9S-a, 9S-b: Outer magnets
10: Fluid communication means
11: Moveable chamber part
12: Hinge
13: Separating wall

In a typical process according to the present invention, the first stream is provided which may be fed with a continuous flow via a first inlet into the precipitation chamber. Simultaneously, the second stream may be fed, also with a continuous flow, via a second inlet into the precipitation chamber. The precipitation chamber may be provided with more than one first inlet for this first stream and more than one second inlet for this second stream. In a next step, the first stream and the second stream are intermixed and said mixture provides a supersaturation and a precipitate results. The mixture of the precipitate and the liquid phase is discharged from the precipitation chamber to the stabilisation chamber, preferably also with a continuous flow. A third stream which contains a second stabilising agent is also fed into the stabilisation chamber where it mixes with the output of the precipitation chamber. The contents of the stabilisation chamber exit through its outlet, preferably into a collecting (or receiving) vessel.

Each mixing chamber (i.e. collectively the precipitation and stabilisation chambers) may have one or more than one outlet. Additionally, in one embodiment, there are no other openings in the mixing chambers besides the inlets and the outlet(s). This means that no solvents, liquids, solutions, particles and the like can enter or exit the mixing chambers except via the specific inlets and the outlet(s). Such chambers are often referred to as “closed type” mixing chambers because they are not open to the air, e.g. in contrast to a beaker that would be an “open type” mixing vessel.

The size of the mixing chambers is dependent on the scale at which the precipitation is performed. On a small scale one typically would use a mixing chamber of volume 0.15 to 100 cm³, for medium scale a mixing chamber of 101 to 250 cm³ and for large scale mixing chamber of more than 250 cm³ may be used. Preferably, the size of the mixing chamber is 1 cm³ to 1 litre. As will be understood, the volume of the mixing chamber is volume without the mechanical stirring means being present. In a preferred embodiment the mixing chamber is a closed type mixing chamber.

Preferably at least one stirrer blade is positioned between the mixing chamber inlets such that it acts as a physical barrier between the incoming streams. In this way the stirrer blade reduces the chance of precipitate formation at the inlets which could otherwise block these inlets. Instead the streams come into contact in a circumferential instead of ‘head-on’ manner.

A device which may be used to perform the process of the present invention is shown schematically in FIG. 1.

This device comprises a precipitation chamber and a stabilisation chamber, as shown on the left and right respectively in FIG. 1, with the outlet of the precipitation chamber connected to the inlet of the stabilisation chamber by a fluid communication means. This device is essentially two of the apparatus disclosed in U.S. Pat. No. 5,985,535, the disclosure in which is expressly incorporated by reference herein, connected by a pipe or hose.

In FIG. 1, the precipitation chamber comprises magnetically driven mechanical stirring means 1P-a and 1P-b, a mixing chamber 3P consisting of a chamber wall 7P having a central axis of rotation facing in top and bottom directions and seal plates 8P which function as tank walls sealing top and bottom opening ends of the chamber wall 7P. The chamber wall 7P and the seal plates 8P are preferably made of non-magnetic materials which are excellent in magnetic permeability if magnetically driven mechanical stirring means is employed which will be elucidated in more detail below. The stirring axes 2P-a and 2P-b are provided with outer magnets 9P-a, 9P-b and are disposed outside at the top and bottom ends of the mixing chamber 3P which are essentially opposite to each other. The outer magnets 9P-a, 9P-b are coupled to mechanical stirring means 1P-a. 1P-b inside the chamber via magnetic forces. Motors (not shown) drive the outer magnets 9P-a and 9P-b in converse directions. By this, mechanical stirring means 1P-a, 1P-b rotate in converse directions in the mixing chamber. The component parts of the stabilisation chamber are analogous to the corresponding parts of the precipitation chamber and therefore we do not need to repeat their detailed description here.

Further, in FIG. 1, the mixing chamber 3P is provided with a first inlet 4P for the first stream, a second inlet 5P for the second stream and a single outlet 6P for exit of the chamber's contents, the outlet 6P being connected to the mixing chamber 3S of the stabilisation chamber by fluid communication means 10 in the form of a pipe or hose. Although inlets 4P and 5P are shown in a diametrically opposed fashion, they may also be aligned in an essentially parallel fashion. As for the shape of the mixing chamber 3P, a cylindrical shape is often used, but rectangular, hexagonal and various other shapes may also be used. Likewise, motors driving outer magnets 9P-a, 9P-b via the axes 2P-a, 2P-b and the mechanical stirring means 1P-a, 1P-b are shown as being disposed at the opposite top and bottom ends of the precipitation chamber 3P, but they may alternatively be disposed at the opposite left and right sides, or may be disposed diagonally, depending on the shape of the mixing chamber. Additionally, the precipitation chamber 3P may comprise more pairs of conversely rotating mechanical stirring means. The component parts of the stabilisation chamber are analogous to the corresponding parts of the precipitation chamber and therefore we do not need to repeat their detailed description here.

In another embodiment, an odd number of magnetically driven mechanical stirring means may be used in one or both of the precipitation and stabilisation chambers, e.g. one, three or five magnetically driven mechanical stirring means. Furthermore, the use of pair wise oriented mechanical stirring means in combination with a single stirring means may lead to even more efficient stirring.

The device according to a second embodiment comprises a precipitation chamber and a stabilisation chamber (the stabilisation chamber shown above the precipitation chamber). Mechanical stirring means 1P and 1S, and shaft 2 are present in mixing chambers 3P and 3S to effect rapid intermixing. The mixing chambers 3P and 3S have walls 7 and are separated by separating wall 13. The mixing chamber 3P of the precipitation chamber has a first inlet 4P for the first stream comprising the organic compound, the inlet 4P being connected to the mixing chamber 3P, a second inlet 5P for the second stream comprising the anti-solvent, the inlet 5P being connected to the mixing chamber 3P. The intermixed first and second streams flow to the mixing chamber 3S of the stabilisation chamber through the channel formed by outlet 6P for the mixing chamber 3P and the inlet 4S to the mixing chamber 3S (this channel constituting a fluid communication means). The mixing chamber 3S of the stabilisation chamber has an inlet for the third stream 5S, an outlet 6S and mechanical stirring means 1S. For illustrative purposes, the mechanical stirring means 1P and 1S are depicted as single stirrer blades, although more than one stirrer blade or other mechanical means which is rapidly movable relative to the chambers 3P and 3S may be used if desired.

The positions as actually depicted in FIG. 2 for inlets 4P and 4S, 5P and 5S for outlets 6P and 6S are also shown only for illustrative purposes. However, other positions of these inlets 4P and 4S, 5P and 5S and for outlets 6P and 6S are feasible and within the scope of the present invention.

In general, a mixing chamber has a bottom part and a top part. Furthermore, one can define a middle line through the mixing chamber dividing the mixing chamber in a bottom part and a top part. Furthermore, one can define the lowest bottom part as 0% height, the middle line as 50% height and the very top as 100% height. Using this general description of the mixing chamber, the inlets 4P and 4S and 5P and 5S preferably are connected at the bottom part of the mixing chamber that is below the middle line, for example below 30% height or 20% height. The outlets 6P and 6S preferably are located at the upper part of the mixing chamber above the middle line, for example above 70% height. The inlets 4P and 5P (and 4S and 5S) may be diametrically opposed to each other. The inlets may also be aligned in an essentially parallel fashion. The inlets may also independently enter the relevant chamber via the lower bottom part. Likewise, outlets 6P and 6S are depicted in FIG. 2 as being positioned at the top of the chambers 3P and 3S, although they may also be positioned in any high portion of a side wall of the relevant mixing chambers and may, for example, be connected by a pipe or hose (not shown) as fluid communication means 10.

The device is preferably provided with or may be connected to a collecting vessel. The collecting vessel preferably comprises a stirring means. Optionally, one or more of the mixing chambers may be surrounded by the collecting vessel. Alternatively, the mixing chambers may be positioned adjacent to or remote from the collecting vessel, depending on user preference. The device and/or the collecting vessel can be provided with a means to control temperature in e.g. the mixing chambers and/or the collecting vessel, respectively. Such control means can for example be used to control the temperature of the streams.

The device may comprise supply tanks (not shown) comprising the fluids which are used to make the streams. The supply tanks may be connected to the relevant chamber by feed lines which can be, for example, hoses or fixed pipes. The transportation of the liquids to the mixing chamber can be done with a continuous flow provided by a pump. The pump can be any pump known in the art as long as the pump can provide a stable flow during a prolonged period of time. Suitable pumps are, for example, plunger pumps, peristaltic pumps and the like.

The shape of the chambers can in principle be chosen freely. Preferably the chambers are rotationally symmetric around a central axis. The chambers can be specified by two identical surfaces. i.e. one top surface and one bottom surface, at a distance x from each other, which surfaces may have any shape, for example from rectangular to dodecagonal or circular with, when applicable, a minimum diameter of Dₘᵢₙ. For example, for a mixing chamber having a square shape, Dₘᵢₙ is the distance between opposite sides. In this embodiment, x can be larger than Dₘᵢₙ and alternatively, x can also be smaller than Dₘᵢₙ. In a further embodiment, the top surface and bottom surface need not to be identical, but one surface can be for example of a smaller size than the other. The chambers can be of the same shape or different shapes. The chambers can be of the same size or different sizes.

In another embodiment the precipitation and stabilisation take place in different parts of the same chamber, that is a precipitation and stabilisation chamber, as illustrated in FIG. 3. In this embodiment the time lag between the intermixing of the first and second streams to form a precipitate of the organic compound in particulate form and the subsequent intermixing of the third stream with the intermixed first and second streams is achieved by positioning the inlet for the third stream downstream from the inlets for the first and second streams.

In FIG. 3, the combined precipitation and stabilisation chamber comprises a mechanical stirring means 1, a mixing chamber wall 7 having a central axis of rotation facing in top and bottom directions. Stirring means 1 is disposed in the centre of the mixing chamber 3, occupying a large % of the volume of the chamber and can be driven via a stirrer axis 2 using a motor (not shown). The inlets 4P, 5P are preferably essentially perpendicular to each other and are positioned upstream from inlet 5S such that the first and second streams intermix, preferably rapidly, before coming into contact with the third stream. However, the positions of inlets 4P and 5P are interchangeable, that is that inlet 4P may enter the mixing chamber 3 via the bottom thereof whereas inlet 5P may enter the mixing chamber 3 via a sidewall. Alternatively, inlet 5P may enter the mixing chamber 3 via the bottom thereof whereas inlet 4P enters the mixing chamber 3 via a sidewall. It is also possible that both inlets 4P and 5P enter through the side wall, in which the angle in a horizontal plane between the inlets can have any value, but is preferably between 90° and 180°. In this embodiment the stirrer axis or shaft 2 is positioned within the outlet 6 of the mixing chamber 3. It is further possible that both inlets 4P and 5P enter via the bottom part of the mixing chamber 3. In a preferred embodiment, inlet 5P via which the second stream enters the chamber is placed at the bottom. In this embodiment unwanted precipitation at the inlet into the chamber is prevented. In any case the inlet 5S for the third stream is placed downstream of the inlets 5P and 4P such that the first and second streams intermix before coming into contact with the third stream.

In the embodiment of FIG. 3 it is also highly preferred that the volume of the stirring means 1 occupies 70% to 99%, more preferably 80% to 98%, especially 90% to 96% of the volume of the mixing chamber. In this way it is easier to ensure that the first and second streams intermix before coming into contact with the third stream. Hence, in this preferred embodiment of the invention the precipitation and stabilisation chamber comprises a mixing chamber having an upstream section 3P (the lower part shown in FIG. 3) where step (d) of the process takes place and a downstream section 3S (the upper part shown in FIG. 3) where step (e) of the process takes place). The precipitation and stabilisation chamber according to the embodiment of FIG. 3 may be constructed from moveable parts as is shown in FIGS. 3A and 3B illustrating a top view of this embodiment of the device. Here, the chamber 3 is formed by two moveable chamber parts 11 that are rotatable around hinges 12. The movable chamber parts 11 interlock around mechanical stirring means 1 (a stirrer blade in the form of a rotatable disc) driven by shaft 2.

A more detailed drawing of the device illustrated in FIG. 2 is shown in FIG. 4. Also this embodiment may be constructed from moveable parts as is shown in FIGS. 3A and 3B.

In FIG. 4, the precipitation and stabilisation chambers comprise mechanical stirring means 1P, 1S in disc form, step (d) of the present process is performed in mixing chamber 3P, step (e) of the present process is performed in mixing chamber 3S, there is a chamber wall 7 and rotatable shaft 2. Also in this embodiment the stirrer axis or shaft 2 is positioned within a single outlet 6P from the mixing chamber 3P where precipitation takes place to the mixing chamber 3S where stabilisation takes place, this outlet 6P also acting as the inlet to the stabilisation chamber. The chamber 3S where stabilisation takes place has an outlet 6S around the rotatable shaft 2. The inlets 4P and 5P are preferably essentially perpendicular to each other. However, also in this embodiment the positions of inlets 4P and 5P are interchangeable and also in this embodiment inlets 4P and 5P may enter the mixing chamber through the side walls or via the bottom part of the mixing chamber 3P. In a preferred embodiment the second stream enters via the bottom part of the mixing chamber 3P.

Additionally, in this embodiment at least, it is also highly preferred that the volume of the mechanical stirring means 1, which in this case have a disc shape 1P, 1S, occupies 70% to 99%, more preferably 80% to 98%, especially 90% to 96% of the volume of the mixing chamber. In this way it is easier to ensure that the first and second streams intermix before coming into contact with the third stream. A mixing chamber having three or more compartments, each compartment being provided with a disk as mechanical stirring means attached to one single axis, can be used. Hence, in this preferred embodiment the device comprises at least two, three, four or more mechanical stirring means in the form of disks being driven by shaft 2, a mixing chamber 3 consisting of a chamber wall 7 having a central axis of rotation facing in top and bottom directions, with inlet 5S for the third stream being positioned downstream from the inlets 5P and 4P to ensure the first and second streams intermix before coming into contact with the third stream.

According to a further aspect of the present invention there is provided a device for the precipitation of an organic compound in particulate form, comprising a precipitation chamber, a stabilisation chamber and a fluid communication means 10 for transporting fluid from the precipitation chamber to the stabilisation chamber, wherein:

(A) the precipitation chamber comprises:
i. a mixing chamber 3P;
ii. an inlet 4P for receiving a first stream into the mixing chamber 3P;
iii. an inlet 5P for receiving a second stream into the mixing chamber 3P;
iv. a mechanical stirring means 1P for intermixing the first and second streams in mixing chamber 3P to form a precipitate of an organic compound in particulate form; and
v. an outlet 6P for releasing the contents of the mixing chamber 3P into the stabilisation chamber via the fluid communication means 10; and

(B) the stabilisation chamber comprises:
i. a mixing chamber 3S;
ii. an inlet 4S for receiving the contents of the mixing chamber 3P from the mixing chamber 3P via a fluid communication means 10 into the mixing chamber 3S;
iii. an inlet 5S for receiving a third stream into the mixing chamber 3S;
iv. a mechanical stirring means 1S for intermixing the contents of the mixing chamber 3S; and
v. an outlet 6S for dispensing the contents of the mixing chamber 3S from the mixing chamber 3S.

In this device the means 1P and 1S for intermixing the contents of the mixing chambers 3P and 3S are preferably mechanical mixing means, for example stirrers which are rotatable within the relevant chamber. The first, second and third streams are preferably as described in the process of the present invention. The intermixing is preferably rapid.

Preferably, all parts of the chambers that are in contact with one or more of the streams are coated with a layer of a material that prevents adhering, fouling, incrustation and such. Preferred materials are those having moisture absorption according to ASTM D 570 at a relative humidity of 50% and a temperature of 23° C. of less than 1%. Suitable examples of such materials include fluorinated alkene polymers and copolymers, e.g. polyvinilydene fluoride polytetrafluoroethylene, and polyacetals, e.g. polyoxymethylene.

At the start of the nucleation, nuclei are surrounded by over-saturated fluid. When two or more of these particles stay in contact for too long, they will be “cemented” together to form an agglomerate. Furthermore, unlike inorganic particles in aqueous media, organic particles are usually not electrically charged and therefore these organic particles do not have a strong electrostatic repulsive mechanism. In the present invention, the drag/shear forces in the mixing chambers imposed on the nuclei by the fluid motion prevents the particles from agglomerating. In one embodiment of this invention, excessive turbulence is used to reduce the inter-particle contact times to values that do not allow agglomeration while the surrounding fluid is still over-saturated.

In the device according to the present invention it was found that a preferred diameter of the mechanical stirring means is at least 50% and more preferably at least 70% and most preferably from 70 to 99% of the smallest diameter of the relevant mixing chamber. Very good results were obtained with a mechanical stirring means which had a diameter of around 90% to 95% of the smallest diameter of the mixing chamber. In another embodiment, very good results were obtained with a mechanical stirring means which had a diameter of 80% to 90% of the smallest diameter of the mixing chamber.

In the device according to the present invention it is highly preferred that the volume of the mechanical stirring means occupies 70% to 99%, more preferably 80% to 98% of the volume of the mixing chamber. In this way it is easier to ensure the first and second streams rapidly intermix before coming into contact with the third stream.

In another embodiment the present invention also provides devices of the general type illustrated in FIG. 3.

According to a further aspect of the present invention there is provided a device for the precipitation and stabilisation of an organic compound in particulate form, comprising:
(i) a mixing chamber;
(ii) mechanical stirring means present in the mixing chamber and occupying 70% to 99% of the volume of said chamber; and
(iii) first, second and third inlets to the mixing chamber for receiving first, second and third streams respectively;
   wherein the third inlet is positioned downstream of the first and the second inlets such that the first and second streams may rapidly intermix before coming into contact with the third stream.

In the present invention, when opposite mechanical stirring means are driven in a mixing chamber (i.e. the shafts rotate in opposite directions), it is preferable to rotate the stirring means at high speed to obtain rapid intermixing. The rotation speed is preferably 1,000 rpm or more, more preferably 3,000 or more, and especially 5,000 rpm or more. A pair of conversely rotating stirring means may be rotated at the same rotating speed or at different rotating speeds. In case of a mechanical stirring means which is symmetrical around an axis, the stirrer speed is preferably at least 500 rpm, for example at least 1,000 rpm or at least 5,000 or even more than 10,000 rpm. Nowadays, mechanical stirrers are commercially available having a stirrer speed of 20,000 rpm and higher. In general, the higher the stirring speed the better the rapid intermixing and therefore there is no particular upper limit for the stirring speed. At very high stirring speeds there is a risk of suspension or solution overheating due to the mixing shear forces and this might cause thermal damage to the precipitated particles or the fluid medium. Such negative effects would set the upper limit of stirring speed for a particular compound or chemical composition unless cooling is applied.

The residence time of the organic compound in the mixing chambers can be varied by, amongst other things, changing e.g. the inflow speeds of the streams, the chamber internal volume or the choice of the type, e.g. shape and size, of the mechanical stirring means. The intensity of intermixing and the positions of the inlets and outlets determine the chances of stirring means being bypassed, causing unmixed fluids to leave the chamber before becoming thoroughly intermixed. A too short residence time of mixed streams in the mixing chambers is undesirable as it may result in uncontrolled nucleation outside the mixing chamber. A too long residence time before coming into contact with the third stream is also undesirable as it may result in excessive agglomeration and growth. Solvent and anti-solvent, together with for example the temperature, can be selected to control the precipitation rate. The nucleation time can for example range from 10⁻⁷ to 10⁻² seconds. Also for compounds not having such a fast nucleation time, the residence times in the precipitation chamber should not be too long, because the efficiency of the precipitation process will be lowered. Furthermore, a long residence time may result in a wide average particle size distribution and larger particles. In practice, the residence time in each of the precipitation and stabilisation chambers (or areas of a combined precipitation and stabilisation chamber) is preferably from 0.1 to 3 seconds. In cases where nucleation proceeds only slowly, e.g. from 10⁻² until 10⁻³ seconds, the conditions are preferably chosen such that the residence time is from 0.1 to 5 seconds, more preferably below 3 seconds and even more preferably below 1 second.

The residence time t may be calculated as follows:

t=V/(a+b)

wherein:
V is the volume of the mixing space in the relevant chamber (cm³);
a and b are the flow rates of the relevant streams into the relevant chamber (cm³/sec).

The process according to the present invention is very suitable for preparation of active pharmaceutical compounds into particulate form, with a small average size and a narrow particle size distribution. Small pharmaceutical particles are very suitable to be used in a medicament. Another advantage of the present invention is that the organic compound often precipitates in an amorphous, non-crystalline form, resulting in enhanced re-dispersion and dissolution rates and solubility.

In another aspect the present invention also provides a process for the manufacture of medicament comprising performing the process of the present invention wherein the organic compound is a pharmaceutically active compound.

Preferably this process further comprises the step of mixing the product of the process with a pharmaceutically acceptable carrier or excipient to give the medicament.

The identity of the carrier or excipient is not crucial provided it is pharmaceutically acceptable. Examples of such carriers and excipients include the diluents, additives, fillers, lubricants and binders commonly used in the pharmaceutical industry.

In a preferred aspect the medicament is in the form of a tablet, troche, powder, syrup, patch, liposome, injectable dispersion, suspension, capsule, cream, ointment or aerosol.

Thus, medicaments intended for oral use may contain, for example, one or more colouring, sweetening, flavouring and/or preservative agents in addition to the product of the presently claimed process (the product of the presently claimed process often being abbreviated herein as simply as “the active ingredient”).

Suitable pharmaceutically acceptable carriers and excipients for a tablet or troche formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl- or propyl-p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance; in either case, using conventional coating agents and procedures well known in the art.

Compositions for oral use may be in the form of hard gelatine capsules in which the active ingredient is mixed with an inert solid diluent, for example, calcium carbonate, calcium phosphate or kaolin, or as soft gelatine capsules in which the active ingredient is mixed with water or an oil such as peanut oil, liquid paraffin, or olive oil.

Aqueous suspensions generally contain the active ingredient either dissolved or in particulate form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxyethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl- or propyl-p-hydroxybenzoate), anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient, optionally together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The medicaments of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The medicaments may also be in the form of a sterile injectable aqueous or oily suspension, which may be formulated according to known procedures using one or more of the appropriate dispersing or wetting agents and suspensing agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the return to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable vehicle or diluent using conventional procedure well known in the art.

Medicaments for administration by insufflation may be in the form of particles made by the presently claimed process, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Medicaments for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on Formulation the reader is referred to Chapter 25.2 in Volume 5 of Comprehensive Medicinal Chemistry (Corwin Hansch; Chairman of Editorial Board), Pergamon Press 1990.

If desired the process may further comprise the step of sterilising the precipitated pharmaceutically active organic compound. The object of the sterilisation is to kill any undesirable bacteria which may cause harm to a patient, particularly if their immune system has been compromised. Typical sterilisation methods include irradiation, filtration through a 0.22 micron sterile filter, heating and treatment with a biocide.

The pharmaceutically active compound referred to in the above further aspects of the present invention may be any of the pharmaceutically active organic compounds mentioned earlier in this specification, especially paclitaxel, fenofibrate or a cyclosporin (e.g. cyclosporin A).

Also the invention provides a medicament obtained by the process of the present invention.

Also the invention provides a method for the treatment of a human or animal comprising administration of a medicament obtained by the process of the present invention. Also the invention provides use of a pharmaceutically active organic compound obtained by the process of the present invention for the manufacture of a medicament for the treatment of cancer.

The invention is now illustrated by the following non-limiting examples in which all parts and percentages are by weight unless otherwise specified.

In these examples, the weight-averaged average particle size D[4,3], median size D50 and D90 were measured with a static light scattering technique using a Malvern Mastersizer 2000. In case the particle size distribution is found to be in the lower micron scale or nano scale, the scatter-intensity-weighted average particle size (Unimodal size), D, was measured using a dynamic light scattering technique, using a Coulter® N4 Plus Submicron Particle Sizer.

The dissolution test results in the following examples were obtained according to the following procedure:

1. Water (20 g) at 37° C. was added to a weighed amount of particulate solid to be redissolved in a glass bottle at start time t=0. The amount of particulate solid was chosen such that it contained 15 milligrams of organic compound (calculated).
2. The mixture of water and particulate solid prepared in stage 1. was subjected to an ultrasonic treatment for 30 seconds using a standard ultrasonic bath to give a suspension.
3. The suspension arising from step 2. was transferred to a glass flat-bottomed flask containing water and the temperature was thermostatically controlled to 37.0±0.5° C., using a propeller stirring blade operated at a gentle stirring rate of 300 rpm to assist dissolution. The amount of water in the flask was chosen so that the final volume of the water was 500 cm³.
4. At regular intervals a sample was taken from the flask, filtered through a 20 nm Al₂O₃ membrane filter (Whatman Anotop® syringe filter) and diluted 2 times on a weight basis with anhydrous ethanol containing 0.1 wt % acetic acid. The filtration was intended to trap any micro- or nanoparticles present in the suspension. The addition of this diluent was intended to prevent any further crystallization of the organic compound. The presence of the acetic acid in the diluent is important in the case of compounds like paclitaxel with a limited chemical stability under alkaline conditions. The acid neutralizes traces of alkaline impurities in the medium and thus improves the chemical stability of the organic compound.
5. The quantitative analysis of the concentration of the organic compound in the filtrate was measured using Ultra Performance Liquid Chromatography (HPLC) with a UV/VIS detector. A high concentration of the organic compound in the filtrate indicated that much of the organic compound had been redissolved (i.e. little of the compound remained in particulate form to be retained by the filter). A lower concentration of the organic compound in the filtrate indicated that less of the organic compound had been redissolved (i.e. more of the compound remained in particulate form and was retained by the filter instead of passing through as a solution in the filtrate).

When measuring particle size distribution using a Mastersizer 2000 particle sizer, it is important that the proper solid refractive index is used. The solid refractive indices of the precipitated organic compounds may be measured according the procedure described in: Saveyn, H., Mermuys, D., Thos, O. and Van der Meeren, P., entitled “Determination of the Refractive Index of Water-Dispersible Granules for use in Laser Diffraction Experiments”, published in Particle and Particle Systems Characterisation, 19 (2002), pages 426-432. For example, by following this procedure the solid refractive index values for pregnenolone, fenofibrate, cyclosporin A and paclitaxel were 1.56, 1.52, 1.50 and 1.51 respectively.

In the following Examples the following chemicals were used:

The chemicals were obtained from Sigma-Aldrich Co., Zwijndrecht, The Netherlands unless specified otherwise:
Paclitaxel from taxus brevifolia, (≧95% by HPLC),
Pregnenolone, ≧98%,
Fenofibrate, ≧99% powder,
Cyclosporin A, BioChemika, ≧98.5% (TLC),
Tetrahydrofuran (THF) biotech grade ≧99.9%, inhibitor-free,
Citric acid, USP grade,
D-Mannitol, USP grade,
The amphiphilic block copolymers.
Anhydrous ethanol 100% DAB, PH.EUR. was obtained from Boom B. V., Meppel, The Netherlands,
Fish gelatine 150 kDa (#1313) was obtained from Norland Products Inc., Cranbury, USA,
Hydrolysed fish gelatine 4.2 kDa (P6132) was obtained from Nitta Gelatine Inc., Japan, The water used was purified by demineralization and filtration techniques on-site.

In all of the following Examples the measured particle size parameters D50. D90 and D[4,3] were stable for at least 15 minutes unless noted otherwise.

In the Examples the particle size distributions, weight averaged size D[4,3], the median size D50 and the D90 (the size which 90% of the particles are below) of resultant particles were measured using a Malvern Mastersizer 2000 unless specified otherwise.

### EXAMPLE 1

In this example the organic compound, paclitaxel, was precipitated in a device comprising a precipitation chamber and a stabilisation chamber, each chamber being of the general type described in U.S. Pat. No. 5,985,535, with the outlet of the precipitation chamber connected to the inlet of the stabilisation chamber by a fluid connection means.

### (A) First Stream—Preparation of First Stock Solution

A first stock solution was prepared comprising the amphiphilic diblock copolymer poly(ethylene glycol) Mn 750 methyl ether-block-polylactide Mn 1000 (20.0 g/l) and the organic compound paclitaxel (20.0 g/l) in tetrahydrofuran solvent. The temperature of the solution was adjusted to 293 K.

### (B) Second Stream—Preparation of Anti-solvent

A second stock solution, of anti-solvent, was prepared by dissolving citric acid (10 g/l) in purified water. The temperature of this anti-solvent was adjusted to 273 K.

### (C) Third Stream—Preparation of a Stabilising Agent Solution

A third stock solution, of stabilising agent, was prepared by dissolving gelatine (91.7 g/l, 4.2 kD molecular weight hydrolysed fish gelatine obtained from Nitta) in water. The solution was then cooled to 273 K.

### (D) The Device

A device according to the invention was made by connecting two magnetically stirred chambers by a hose, each constructed as described in U.S. Pat. No. 5,985,535, FIG. 1. The first chamber was a precipitation chamber and the second chamber was a stabilisation chamber, with the outlet of the first chamber connected to an inlet of the second chamber using the hose as fluid communication means. Each chamber was of the closed type, cylindrical with an internal volume of 1.5 cm³ (prior to incorporation of the mechanical stirring means), two spaced inlets, a pair of magnetically driven stirrer blades as mechanical stirring means and one outlet. The mechanical stirring means in the form of stirrer blades had diameters of 83% of the chamber diameter. The volume of the mechanical stirring means was 0.8 cm³.

### (E) The Process

The device was filled with liquid by pumping a stream of the second stock solution (anti-solvent) through it at a rate of 100 cm³/min. The stirrer blades in the precipitation and stabilisation chambers were operated at 6,000 RPM in opposite directions.

When the device was full, the first stream (containing the organic compound) was pumped into the precipitation chamber at a rate of 20 cm³/min and the third stream (containing the second stabilising agent) was pumped into the stabilisation chamber at a rate of 120 cm³/min. The temperature in the precipitation chamber was 1° C.

The initial output of the device was discarded until its composition became constant, after which the three streams were continuously pumped into the device and the output from the stabilisation chamber was collected in a batchwise manner. At the end of the manufacture the device was flushed through with solvent and the washings retained for recycling.

### (F) Results—Particle Size Distribution

The particles were found to have a unimodal particle size distribution, a D[4,3] of 105 nm, a D50 of 99 nm and a D90 of 148 nm.

The unimodal size as measured by a Coulter® N4 Plus Submicron Particle Sizer was 65 nm.

### (G) Results—Particle Form

The physical form of the paclitaxel particles was measured using X-ray powder diffraction method (“XRPD”) before the process (i.e. the starting material) and again after the product of the process had been freeze dried.

The XRPD spectra indicated that prior to the process the paclitaxel had crystal structure ordering. However the freeze dried product obtained by the process was in the more desirable amorphous form.

### (H) Results—Storage Stability Test

The average size of the particulate suspension, D, was measured using a Coulter N4 Plus apparatus both before and after standing in a vessel at 0° C. for 40 hours. Over this long period there was no significant change in particle size. The initial particle size D was 66 nm (triple measurement, 90°, 300 seconds) and the final particle size was 71 nm. The particle size distribution was narrow with a polydispersity index of 0.08 initially and 0.07 after 40 hours.

### (I) Results Redispersibility

After freeze drying the particles were dispersed in water at room temperature and subjected to 30 seconds ultrasound. The resulting suspension was inspected visually and by means of a size measurement using the Coulter® N4 Plus Submicron Particle Sizer. The result showed that the particles had redispersion in water readily to an average size of 130 nm.

### (J) Results—Dissolution Rate

The freeze dried particles of paclitaxel obtained by the claimed process dissolved in water at 37° C. much better than the untreated particles of paclitaxel (about 7 times higher concentration 0.5 hrs after dissolving).

### EXAMPLE 2

The method of Example 1 was repeated except that in place of 4.2 kD molecular weight hydrolysed fish gelatine in the third stock solution there was used an identical weight of deep-sea fish gelatine (150 kD molecular weight, from Norland).

### (A) Results—Particle Size Distribution

The particles were found to have a unimodal particle size distribution, a D[4,3] of 109 nm, a D50 of 103 nm and a D90 of 150 nm.

The unimodal size as measured by a Coulter® N4 Plus Submicron Particle Sizer was 88 nm. The particle size distribution was narrow with a polydispersity index of 0.3.

### (B) Results—Storage Stability Test

An assessment lasting 2 hours showed no signs of particle size instability.

### (C) Results—Redispersibility

After freeze drying the particles were dispersed in water at room temperature and subjected to 30 seconds ultrasound. The resulting suspension was inspected visually and by means of a size measurement using a Coulter® N4

Plus Submicron Particle Sizer. The result showed that the particles had redispersed in water readily to an average size of 206 nm.

### (D) Results—Dissolution Rate

The freeze dried particles of paclitaxel obtained by the claimed process dissolved in water at 37° C. much better than the untreated particles of paclitaxel (about 9 times higher concentration 0.5 hrs after dissolving).

### EXAMPLE 3

The method of Example 1, steps (A) to (E), were repeated except that (A) the first stock solution comprised the amphiphilic diblock copolymer poly(ethylene glycol) Mn 2000 methyl ether-block-polylactide Mn 2000 (60.0 g/l) and the organic compound paclitaxel (60.0 g/l) in tetrahydrofuran as solvent at 293 K; (B) the second stream stock solution comprised 5 g/l of citric acid in water; and (C) the third stock solution was prepared by dissolving deep sea fish gelatine (150 kD molecular weight) in water (45.9 g/l).

### (F) Results—Particle Size Distribution

The particles were found to have a unimodal particle size distribution, a D[4,3] of 235 nm, a D50 of 127 nm and a D90 of 237 nm.

The unimodal size as measured by a Coulter® N4 Plus Submicron Particle Sizer was 170 nm. The particle size distribution was narrow with a polydispersity index of 0.3 and did not change much during storage.

### (G) Results—Redispersibility

After freeze drying the particles were dispersed in water at room temperature and subjected to 75 seconds ultrasound. The resulting suspension was inspected visually and by means of a size measurement using the Coulter® N4 Plus Submicron Particle Sizer. The particles readily redispersed in water to a unimodal size of 247 nm with a polydispersity of 0.3.

### (H) Results—Dissolution Rate

The resultant freeze dried particles of paclitaxel obtained in this Example dissolved in water at 37° C. much better than the untreated particles of paclitaxel (about 4 times higher concentration 0.5 hrs after dissolving).

### EXAMPLE 4

The method of Example 1, steps (A) to (E), was repeated except that (A) the first stock solution consisted of fenofibrate (20 g/l) and the amphiphilic diblock copolymer poly(ethylene glycol) Mn 750 methyl ether-block-polylactide Mn 1000 (4.5 g/l) in ethanol solvent at 293 K; (B) the second stream stock solution comprised 5 g/l of citric acid in water; and (C) the third stream stock solution was prepared by dissolving deep sea fish gelatine (150 kD molecular weight) in water (45.9 g/l).

### (F) Results—Particle Size Distribution

The particles were found to have a unimodal particle size distribution, a D[4,3] of 131 nm, a D50 of 122 nm and a D90 of 206 nm.

The unimodal size as measured by a Coulter® N4 Plus Submicron Particle

Sizer was 196 nm. The particle size distribution was narrow with a polydispersity index of 0.27 and did not change much during storage.

### (G) Results—Redispersibility

After freeze drying the particles were dispersed in water at room temperature and subjected to 30 seconds ultrasound. The resulting suspension was inspected visually and by means of a size measurement using the Coulter® N4 Plus Submicron Particle Sizer. The particles readily redispersed in water to a unimodal size of 500 nm with a polydispersity of 0.7.

### (H) Results—Dissolution Rate

The resultant freeze dried particles of fenofibrate obtained in this Example dissolved in water at 37′C much better than the untreated particles of fenofibrate.

### EXAMPLE 5

The method of Example 1, steps (A) to (E), were repeated except that (A) the first stock solution consisted of cyclosporin A (10 g/l) and the amphiphilic diblock copolymer poly(ethylene glyco)) Mn 750 methyl ether-block-polylactide Mn 1000 (10.0 g/l) in tetrahydrofuran solvent at 293 K; (B) the second stream stock solution comprised 5 g/l of citric acid in water; and (C) the third stream stock solution was prepared by dissolving deep sea fish gelatine (150 kD molecular weight) in water (45.9 g/l).

### (F) Results—Particle Size Distribution

There were micron-sized particles detected in the batch accounting for less than 5% (m/m) of the weight of particles but these are believed to be aggregates of loosely packed primary particles.

The D[4,3] was 750 nm, the D50 was 124 nm and the D90 was 224 nm.

The unimodal size as measured by a Coulter® N4 Plus Submicron Particle Sizer was 109 nm. The particle size distribution was narrow with a polydispersity index of 0.3 and did not change much during storage.

### (G) Results—Redispersibility

After freeze drying the particles were dispersed in water at room temperature and subjected to 240 seconds ultrasound. The resulting suspension was inspected visually and by means of a size measurement using the Coulter® N4 Plus Submicron Particle Sizer. The particles readily redispersed in water to a unimodal size of 157 nm with a polydispersity of 0.2.

### (H) Results—Dissolution Rate

The resultant freeze dried particles of cyclosporin A obtained in this Example dissolved in water at 37° C. much better than the untreated particles of cyclosporin A.

### COMPARATIVE EXAMPLES 1 TO 8

In the Comparative Examples 1 to 8 the general method of Example 1 was repeated except that the single chamber device of U.S. Pat. No. 5,985,535 was used and the step (C) (introducing a third stream containing a second stabilising agent) was omitted. In these Comparative Examples PEG refers to poly(ethylene glycol) methyl ether. PLA refers to polylactide methyl ether, polycaprolactone refers to poly(ε-caprolactone) methyl ether. The more detailed process conditions are described in the fourth column. Mixing Chamber B was a cylindrical mixing chamber of volume 1.5 cm³, with stirrer blades at opposite faces occupying 83% of the chamber diameter and rotating at 6000 rpm in opposite directions.

Redispersibility refers to how well a freeze dried sample resumes its freshly prepared size when re-introduced into water.

| | | | Detailed | | | | Storage |
|---|---|---|---|---|---|---|---|
| Comp | | Second Stream | Process | D[4,3] | D50 | D90 | Stability/ |
| Ex. | First Stream | (anti-solvent) | Conditions | (nm) | (nm) | (nm) | Redispersibility |
| 1 | Fenofibrate | Water at 293K | 1^{st} stream | Unimodal | | | Unstable. |
| | (20 g/l) and PEG | | 10 cm³/min | 123 | 111 | 206 | Within 5 min at |
| | Mn 750, PLA Mn | | 2^{nd} stream | | | | 20° C. the D50 |
| | 1000 (4.4 g/l) in | | 110 cm³/min | | | | increased to 5.1 |
| | ethanol at 293K | | | | | | μm and D90 to |
| | | | | | | | 9.7 μm. |
| 2 | Paclitaxel (10 g/l) | Water at 273K | 1^{st} stream | Bimodal | | | Unstable. |
| | and PEG Mn 5000, | | 20 cm³/min | 1000 | 296 | 482 | Within 7 min at |
| | PLA Mn 5000 (10 g/l) | | 2^{nd} stream | | | | 1° C., the D50 |
| | in THF at 293K | | 100 cm³/min | | | | increased to |
| | | | | | | | 24.1 μm, the |
| | | | | | | | D90 increased |
| | | | | | | | to 55.1 μm and |
| | | | | | | | the D[4,3} to |
| | | | | | | | 28.7 μm. |
| 3 | Paclitaxel (10 g/l) | Gelatine | Same as | Unimodal | | | Unstable. |
| | and PEG Mn ~5000, | (20 g/l, | Comparative | 121 | 114 | 174 | Within 8 min at |
| | polycaprolactone | 4.2 kD, | Example 2. | | | | 1° C., the D50 |
| | Mn ~32,000 | hydrolysed | | | | | increased to |
| | (10 g/l) in THF at | fish) in | | | | | 17.9 μm, the |
| | 293K | water at | | | | | D90 increased |
| | | 273K. | | | | | to 45.3 μm and |
| | | | | | | | the D[4,3} to |
| | | | | | | | 51.3 μm. |
| 4 | Paclitaxel (10 g/l) | Water at 273K | Same as | Trimodal | | | Not good due to |
| | and PEG Mn | | Comparative | 153 | 118 | 212 | crystal formation. |
| | 350, PLA Mn | | Example 2 | | | | |
| | 1000 (10 g/l) in | | but in mixing | | | | |
| | THF at 293K | | chamber B. | | | | |
| 5 | Paclitaxel (10 g/l) | Water at 273K | Same as | Unimodal | | | Stable for at |
| | and PEG Mn 750, | | Comparative | 127 | 119 | 193 | least 15 min. Poor |
| | PLA Mn 1000 (10 g/l) | | Example 4. | | | | redispersibility |
| | in THF at 293K | | | | | | in water, resulting |
| | | | | | | | in visible macro- |
| | | | | | | | scopic particles. |
| 6 | Cyclosporin A | 1 wt % citric | Same as | Unimodal | | | Stable for at |
| | (10 g/l) and PEG | acid in water | Comparative | 132 | 114 | 187 | least 30 min. |
| | Mn 750, PLA Mn | at 273K | Example 4. | | | | Very poor |
| | 1000 (10 g/l) in | | | | | | redispersibility |
| | THF at 293K | | | | | | in water, so bad |
| | | | | | | | that particle |
| | | | | | | | size could not |
| | | | | | | | be measured. |
| 7 | Pregnenolone | 4 wt % 4.2 kD, | Same as | Bimodal | | | Moderate |
| | (34 g/l) and PEG | hydrolysed non- | Comparative | 1820 | 1360 | 4580 | storage stability |
| | Mn 750, PLA Mn | gelling fish | Example 1. | | | | and |
| | 1000 (4.4 g/l) in | gelatine mw 4.2 kDA | | | | | redispersibility. |
| | ethanol at 323K | in water at 273K. | | | | | |
| 8 | Same as | 1 wt % citric | Same as | 118 | 111 | 170 | Redispersibility |
| | Comparative | acid in water | Comparative | | | | moderate, with |
| | Example 5. | at 273K | Example 4. | | | | some particles |
| | | | | | | | visible in the |
| | | | | | | | suspension and |
| | | | | | | | a wide particle |
| | | | | | | | size distribution |
| | | | | | | | with an average |
| | | | | | | | size D of 280 nm. |

### COMPARATIVE EXAMPLE 9

### Continuously Stirred, Open Tank

A solution of pregnenolone in ethanol (34 g/l) was added over 45 seconds, with stirring, to a tank containing pure water as precipitation agent (1500 cm³). The rate of addition was 1000 cm³/min. The stirrer rotational speed was 750 rpm. Turbidity was observed immediately after the addition started.

The precipitate was found to have a wide particle size distribution, including many particles of 10 μm edge length or more. The particles had a D50 of 14.59 μm and a D90 of 36.24 μm. Stability measurements were not performed due to the undesirable initial size.

### COMPARATIVE EXAMPLE 10

### Chamber—No Stabilizer

The method of Comparative Example 1 was repeated except that in place of the fenofibrate there was used pregnenolone in ethanol (34 g/l) and water was used as the precipitation agent. No stabilizer was used. The solvent solution and the precipitation agent were fed into the chamber at 275 K. The total batch addition time to make 100 cm³ of product was 50 seconds. The resultant particles were discharged from the chamber through the outlet port and collected. The particles had a D50 of 9.17 μm and a D90 of 18.72 μm.

### Summary of Results:

| Example: | 1 | 2 | 3 | 4 | 5 |
|---|---|---|---|---|---|
| Method | As claimed | As claimed | As claimed | As claimed | As claimed |
| Organic compound | paclitaxel | paclitaxel | paclitaxel | fenofibrate | Cyclosporin A |
| Particle size distribution | Unimodal | Unimodal | Unimodal | Unimodal | Bi-modal* |
| D50 (nm) | 99 | 103 | 127 | 122 | 124 |
| D90 (nm) | 148 | 150 | 237 | 206 | 217 |
| Storage Stability | Good | Good | Good | Good | Good |
| Redispersibility after | Easy | Easy | Easy | Easy | Easy |
| freeze drying | | | | | |

*Larger “particle” fraction is believed to be reversibly aggregated primary particles.

### COMPARATIVE EXAMPLES

| Comparative Example: | 1 | 2 | 3 | 4 | 5 |
|---|---|---|---|---|---|
| Method | Single Stabiliser | Single Stabiliser | Two stabilizers | Single Stabiliser | Single Stabiliser |
| Organic compound | fenofibrate | paclitaxel | paclitaxel | paclitaxel | paclitaxel |
| Particle size distribution | Unimodal | Bi-modal | Unimodal | Tri-modal | Unimodal |
| D50 (nm) | 111 | 296 | 114 | 118 | 119 |
| D90 (nm) | 206 | 482 | 174 | 212 | 193 |
| Storage Stability | Bad | Bad | Very Bad | Bad | Good |
| Redispersibility after | — | — | — | — | Bad |
| freeze drying | | | | | |
| Comparative Example: | 6 | 7 | 8 | 9 | 10 |
| Method | Single Stabiliser | Single Stabiliser | Single Stabiliser | Stirred tank | Single Stabiliser |
| | | | | Single Stabiliser | |
| Organic compound | cyclosporin A | pregnenolone | paclitaxel | pregnenolone | pregnenolone |
| Particle size distribution | Unimodal | Bimodal | Unimodal | Very wide | Narrower than |
| | | | | distribution | Comp Ex 9 |
| D50 (nm) | 114 | 1,360 | 111 | 14,590 | 9,170 |
| D90 (nm) | 187 | 4,580 | 170 | 36,220 | 18,720 |
| Storage Stability | Good | Bad | Moderate | — | — |
| Redispersibility after | Bad | | Moderate | — | — |
| freeze drying | | | | | |

1.-32. (canceled)

33. A process for the precipitation of an organic compound in particulate form comprising the steps:
(a) providing a first stream comprising an organic compound and a solvent for the organic compound;
(b) providing a second stream comprising an anti-solvent for the organic compound;
(c) providing a third stream comprising an amphiphilic copolymer as second stabilising agent;
(d) intermixing the first and second streams to form a precipitate of the organic compound in particulate form; and
(e) following step (d), intermixing the third stream with the intermixed first and second streams;
wherein the first and/or the second stream comprises an amphiphilic block copolymer as first stabilising agent.

34. A process according to claim 33 wherein the said amphiphilic copolymer comprises a gelatine having a molecular weight of at least 2 kDa and showing no gelling properties when stored as a 2 wt % solution in water at 10° C. for 4 hours.

35. A process according to claim 34 wherein the gelatine is recombinant gelatine.

36. A process according claim 33 wherein the first stabilising agent comprises a poly(ethylene glycol) monoether polylactide amphiphilic block copolymer.

37. A process according claim 33 wherein the second stream further comprises citric acid as peptising agent.

38. A process according to claim 33 wherein:
(i) the first stream comprises a water-miscible organic solvent for the organic compound and the first stabilising agent comprises an amphiphilic block copolymer;
(ii) the second stream comprises water and optionally citric acid; and
(iii) the third stream comprises water and the second stabilising agent comprises a gelatine.

39. A process according to claim 33 wherein the solvent comprises one or more water-miscible organic solvents.

40. A process according to claim 39 wherein the anti-solvent comprises water.

41. A process according to claim 33 wherein the intermixing in steps (d) and (e) each independently is performed for 0.1 milliseconds to 5 seconds.

42. A process according to claim 33 wherein step (d) takes place in a closed type mixing chamber and step (e) takes place in a closed type mixing chamber which is the same chamber or a different chamber from that used in step (d).

43. A process according to claim 33 wherein said intermixing is performed in a chamber fitted with at least one mechanical stirring means having a diameter of 70% to 99% of the smallest diameter of the mixing chamber.

44. A process according to claim 33 wherein the organic compound is a pharmaceutically active compound.

45. A process according to claim 44 wherein the pharmaceutically active compound is paclitaxel, fenofibrate or a cyclosporine.

46. A process according to claim 44 wherein the solvent comprises one or more water-miscible organic solvents and the anti-solvent comprises water.

47. A process for the manufacture of medicament comprising performing a process according to claim 44 and mixing the product thereof with a pharmaceutically acceptable carrier or excipient to give the medicament.

48. A device for the precipitation of an organic compound in particulate form, comprising a precipitation chamber, a stabilisation chamber and a fluid communication means for transporting fluid from the precipitation chamber to the stabilisation chamber, wherein:
(A) the precipitation chamber comprises:
(i) a closed type mixing chamber;
(ii) an inlet for receiving a first stream into the mixing chamber;
(iii) an inlet for receiving a second stream into the mixing chamber;
(iv) a mechanical stirring means for intermixing the first and second streams in the mixing chamber to form a precipitate of an organic compound in particulate form; and
(v) an outlet for releasing the contents of the mixing chamber into the stabilisation chamber via the fluid communication means; and
(B) the stabilisation chamber comprises:
(i) a closed type mixing chamber;
(ii) an inlet for receiving the contents of the mixing chamber from the mixing chamber via a fluid communication means into the mixing chamber;
(iii) an inlet for receiving a third stream into the mixing chamber;
(iv) a mechanical stirring means for intermixing the contents of the mixing chamber; and
(v) an outlet for dispensing the contents of the mixing chamber from the mixing chamber.

49. A device according to claim 48 wherein the mechanical stirring means occupy 70% to 99% of the volume of the mixing chamber.
